(19) United States
(12) Patent Application Publication
(10) Pub. No.: US 2016/0051773 A1
(43) Pub. Date: Feb. 25, 2016

(54) SAFETY CAP FOR DISPOSABSLE SYRINGE

(71) Applicant: Nyeon Sik EUM, Daegu (KR)

(72) Inventor: Nyeon Sik EUM, Daegu (KR)

(21) Appl. No.: 14/779,062

(22) PCT Filed: Jun. 20, 2013
(86) PCT No.: PCT/KR2013/005458

§ 371 Date: Sep. 22, 2015

(30) Foreign Application Priority Data
Mar. 22, 2013 (KR) 10-2013-0030761

(51) Int. Cl. A61M 5/32

(52) U.S. Cl. CPC A61M 5/3216

(57) ABSTRACT

The present invention relates to a safety cap for a disposable syringe. In particular, the safety cap for covering a needle of the syringe has a cutout insertion portion formed at one side thereof and a ball provided at the inner upper end of the cutout insertion portion, so that it is possible to couple the needle of the syringe to the safety cap by inserting the needle into the ball and then rotating the needle about the ball. Further, the safety cap has a fitting groove formed at the lower end portion thereof, so that the safety cap can be coupled to and covered on the upper portion of the used syringe through the cutout insertion portion from the lateral side of the syringe, and be then forcibly coupled to the upper portion of the syringe through the fitting groove. Therefore, the safety cap can prevent a user, after using the syringe, from having a wound by being pierced by the needle or from being infected by germs through the wound.

### CROSS-REFERENCE TO RELATED APPLICATIONS

This application claims priority to and the benefit of Korean Patent Application No. 10-2013-030761 filed in the Korean Intellectual Property Office on Mar. 22, 2013, the entire contents of which are incorporated herein by reference.

### TECHNICAL FIELD

The present invention relates to a safety cap for a disposable syringe, and more specifically to a safety cap for a disposable syringe configured such that a cutout insertion portion is formed at one side of the safety cap covering a needle of the syringe, a ball is provided at the inner upper end of the cutout insertion portion so that a needle is coupled to the safety cap by inserting the needle into the ball and rotating it, and a fitting groove is formed at the lower end portion so that the safety cap is coupled to and covered on the upper portion of the syringe through the cutout insertion portion from the lateral side of the syringe after use and then is forcible coupled to the upper portion of the syringe through the fitting groove, thereby preventing a user, after using the syringe, from having a wound by being pierced by the needle or from being infected by germs through the wound.

### BACKGROUND ART

Recently, there was an accident where a worker in a renowned national university hospital was pricked by a needle used on an HIV patient. Also, oftentimes, there are accidents where nurses get pricked by a needle while disposing disposable syringes after use.

Also, as there are many cases where doctors or nurses get their fingers pricked by the tip of a needle by carelessly pressing down the cap protecting the needle in the wrong direction while covering the cap downwardly in the vertical direction from the tip of the needle after using a disposable syringe, the Korean government is seeking enactment to make the use of safety syringes in hospitals compulsory. However, in practice, a considerable number of hospitals are avoiding the use of safety syringes for cost reasons.

For this reason, recently, more hospitals are giving orders to dispose syringes without covering the needle with a safety cap after use, and accordingly examples of secondary damages on workers such as cleaners are gradually increasing.

In order to solve the above problem, currently as a safety syringe, a syringe where a safety device protecting the needle is coupled to the surface of the syringe and the syringe is disposed by operating the needle protecting device after use, or a syringe where the needle is inserted into the syringe, or a syringe with a modified shape, is generally used.

However, considering the cost of disposable syringes, these safety syringes have been neglected because they are a number of times more expensive than the existing syringes. However, recently, many efforts have been made to make the use of safety syringes compulsory for those handling diseases that may be infected through blood.

As background art of the present invention where the above problem of safety syringes has been improved, as illustrated in FIG. 8, there is provided a cap attached syringe formed with an elastic means (e.g., spring hinge) where between a cap fitting portion 50 and a cap main body 70, the cap main body 70 is rotated with respect to a hinge portion 60, so that, with regard to the use position exposing a needle 20, force is applied in a direction fitting the cap main body 70 in the cap fitting portion 50, and simultaneously, when counter-rotating the cap main body 70 with respect to the hinge portion 60, force is applied to the cap main body 70 towards the direction where the needle 20 is placed within the receiving portion.

However, although the conventional cap attached syringe has a cap fit into the syringe by having the cap inserted from the lateral side of the needle, since the cap is attached to the syringe, there is inconvenience in use because the cap is always attached even when using the syringe. Further, although the syringes should be manufactured at low cost considering the property of disposable syringes, since the syringes need to be manufactured having the cap attached to the syringe, there is limitation in manufacturing the syringes at low cost.

### SUMMARY OF INVENTION

The present invention was derived to improve the overall problems discussed in the above. It is an object of the present invention to provide a safety cap for a disposable syringe formed with a cutout insertion portion from the lateral side of the safety cap covering the needle at the upper portion of the syringe, and a fitting groove at the lower end portion of the safety cap formed with the cutout insertion portion, so as to lift up the safety cap in the vertical direction and separate the safety cap from the syringe when using the syringe, and after using the syringe, cover the safety cap from the lateral side of the needle of the syringe through the cutout insertion portion and fit the safety cap in the upper neck portion of the syringe through the fitting groove, which has the safety of the conventional safety syringe, while allowing the manufacture of cheap forms of safety caps for safety syringes.

That is, the present invention aims to provide a safety cap for a disposable syringe that allows safe disposal of disposable syringes while allowing fingers to avoid the tip of the needle by coupling the safety cap through one-touch coupling from the lateral side, not by a coupling method vertically covering the safety syringe like conventional safety caps.

It is another object to provide a safety cap for a disposable syringe whose cap is fit being forcibly coupled to the upper portion of the syringe by forming the fitting groove of the cap to have almost the same size as the protrusion formed around the neck portion of the upper portion of the syringe.

It is yet another object to provide a safety cap for a disposable syringe formed with a cutout insertion portion at one side of the safety cap, and provided with a ball at the inner upper end of the cutout insertion portion so that the needle is coupled to the safety cap by inserting the needle into the ball and rotating it, thereby preventing a user, after using the syringe, from having a wound by being pierced by the needle or from being infected by germs through the wound.

It is still yet another object to provide a safety cap for a disposable syringe where the inside of the ball is empty, so that when inserting the needle into the ball, part of the ball is inserted into the needle, thereby blocking the tip of the needle and preventing reuse of needle.

In order to achieve the above objects, the safety cap for a disposable syringe of the present invention coupled to an upper end portion of the main body of the syringe for covering the upper end portion is characterized in that the safety cap is formed with a cutout insertion portion cut out in the longitudinal direction so as to have an upper portion of the syringe including a needle inserted from the lateral side, and a fitting groove with a predetermined width in the longitudinal direction is formed by being partially cut out in the circumferential direction at a lower end portion formed with the cutout insertion portion.

Also, preferably, the safety cap for a disposable syringe is lifted up in the vertical direction and separated from the syringe when using the syringe, and after using the syringe, the safety cap covers the needle by having an upper portion of the syringe inserted from the lateral side through the cutout insertion portion and simultaneously a protrusion formed around the upper portion of the syringe is inserted and fit into a fitting groove formed at the lower end portion of the safety cap.

Also, preferably, the width in the longitudinal direction of the fitting groove of the safety cap is formed be slightly different from the thickness of the protrusion of the syringe, so that the protrusion is inserted in the fitting groove and the safety cap is forcibly coupled to the upper portion of the syringe.

Also, the safety cap is characterized by being provided with a ball at the inner upper end portion, and formed with a cutout insertion portion in communication with the outside so that the needle is coupled to the upper end portion of the main body of the syringe by inserting the needle into the ball and rotating the ball.

Also, preferably, the ball is made from a silicone or rubber material, empty in the inside.

Also, preferably, the safety cap for a disposable syringe is formed in the shape of a cone or a prismoid, and the lower end portion is formed to be greater than the diameter of the upper end portion of the main body of the syringe.

Also, preferably, the safety cap for a disposable syringe is formed in a color of any one of green, blue, yellow, grey and brown, which gives psychological stability.

According to the safety cap for the disposable syringe of the present invention, since the needle of the syringe is covered after use by inserting the needle from the lateral side through the cutout insertion portion formed in the safety cap, there are effects such that there is hardly any danger of accidents having fingers pricked by the needle, and the syringe can be safely disposed after use.

Also, since the safety cap is firmly fit into the syringe after use by the fitting groove formed in the safety cap, there is an effect that the needle can be safely protected by the safety cap even when the needle is separated from the syringe.

Also, because safety caps have a simple structure considering the property of disposable syringes which have to be manufactured at low cost, there is an effect that the manufacturing cost can be greatly reduced as compared to the conventional safety syringe.

Also, since a cutout insertion portion is formed at one side of the safety cap for the disposable syringe, and a ball is provided at the inner upper end of the cutout insertion portion so that a needle of the syringe is coupled to the safety cap by inserting the needle of the syringe into the ball and rotating it, there is an effect of preventing a user, after using the syringe, from having a wound by being pierced by the needle or from being infected by germs through the wound.

Also, since the inside of the ball is empty, when inserting the needle of the syringe into the ball, part of the ball is inserted into the inside of the needle of the syringe, and thus there is an effect of blocking the tip of the needle and preventing reuse of needles.

### BRIEF DESCRIPTION OF THE DRAWINGS

FIG. 1 is a perspective view of a safety cap for a disposable syringe according to an embodiment of the present invention;

FIG. 2 is a side view of a safety cap for a disposable syringe according to an embodiment of the present invention;

FIG. 3 is a state diagram of the combination between a safety cap for a disposable syringe according to an embodiment of the present invention and a syringe before use;

FIG. 4 is a state progress diagram of the combination between a safety cap for a disposable syringe according to an embodiment of the present invention and a syringe after use;

FIG. 5 is a state diagram of the combination between a safety cap for a disposable syringe according to an embodiment of the present invention and a syringe after use;

FIG. 6 is a front view of a safety cap for a disposable syringe before using the syringe according to another embodiment of the present invention;

FIG. 7 is a state diagram of the use of a safety cap for a disposable syringe according to another embodiment of the present invention; and

FIG. 8 is a side view of a safety syringe before combining the conventional safety cap with a syringe.

### BEST MODE FOR CARRYING OUT THE INVENTION

Hereinafter, preferable embodiments of the safety cap for the disposable syringe according to an embodiment of the present invention are explained in detail with reference to the accompanying drawings. The present invention is not limited to such embodiments disclosed and may be modified variously in many different forms. The embodiments are provided to complete the disclosure of the present invention and to allow those having ordinary skill in the art to understand the scope of the present invention.

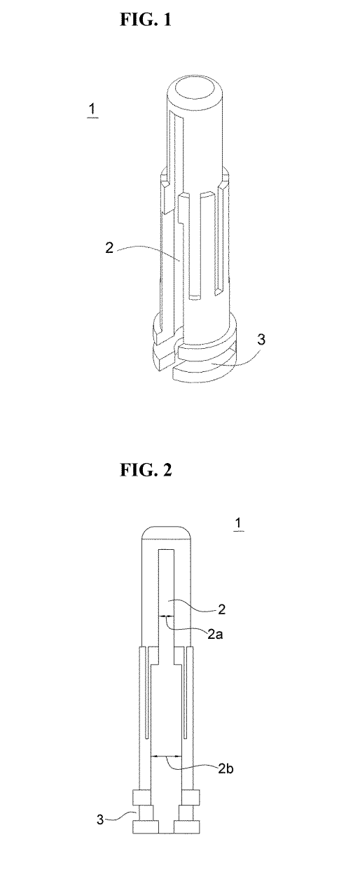

FIG. 1 is a perspective view of a safety cap for a disposable syringe according to an embodiment of the present invention, and FIG. 2 is a side view of a safety cap for a disposable syringe according to an embodiment of the present invention.

As illustrated in FIG. 1 and FIG. 2, the safety cap for the disposable syringe according to an embodiment of the present invention includes a cutout insertion portion 2 formed at the lateral side of the safety cap 1 and a fitting groove 3 formed at the lower end portion.

The cutout insertion portion 2 is formed with an open groove lengthily cut out along the longitudinal direction from the lateral side of the safety cap 1, and it would be sufficient for the cutout insertion portion 2 cut out in the width direction to have a size where the upper portion of the syringe including the needle may fit. Referring to the shape of the cutout insertion portion 2 in detail, it would be preferable to configure the length 2a of the portion where the needle of the syringe is located cut out in the width direction to be shorter than the length 2b of the portion where the neck in the upper portion of the syringe is located below the needle of the syringe cut out in the width direction, so as to protect the needle of the syringe more securely.

The fitting groove 3 is formed in a C-shaped groove cut out along the circumferential direction of the lower end portion of the safety cap 1 formed with a cutout insertion portion 2 leaving only part of it, and the width in the longitudinal direction of the groove is configured to be almost the same as the protrusion of the upper portion of the syringe which will be explained in the following.

Hereinafter, an example of using a disposable safety syringe according to an embodiment of the present invention configured as above will be explained.

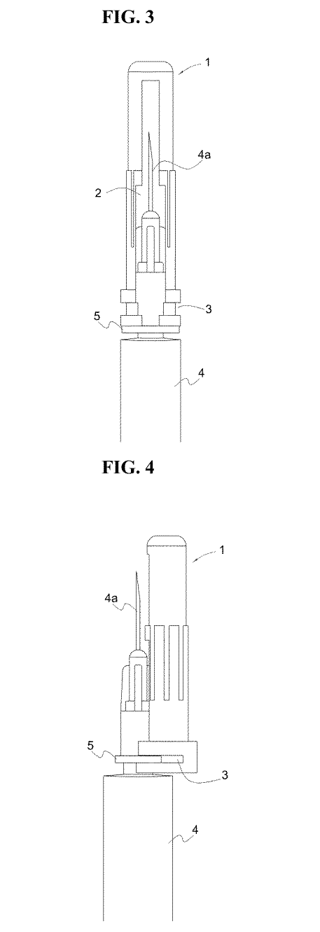

FIG. 3 is a state diagram of the combination between a safety cap for a disposable syringe according to an embodiment of the present invention and a syringe before use.

As illustrated in FIG. 3, the safety cap 1 for the disposable safety syringe according to an embodiment of the present invention is coupled to an upper portion of the syringe 4 with a needle 4a, and fit onto a protrusion 5 formed in the neck portion of the upper portion of the syringe 4. When using the syringe 4 in a state before using the syringe 4, the syringe is used after vertically lifting up the safety cap 1 of the present invention and separating it from the upper portion of the syringe 4.

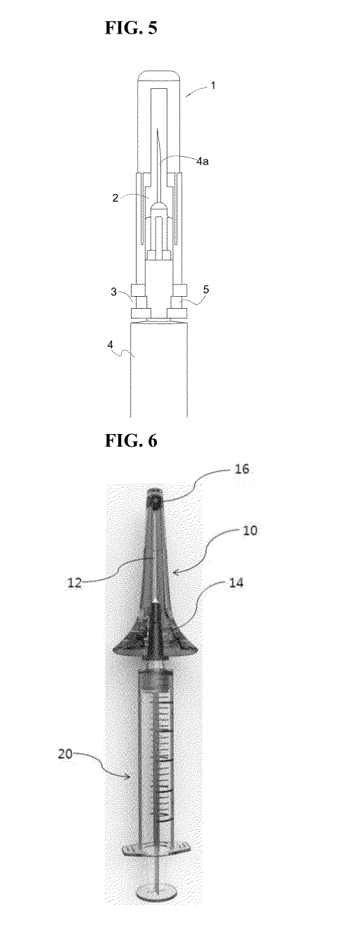

FIG. 4 is a state progress diagram of the combination between a safety cap for a disposable syringe according to an embodiment of the present invention and a syringe after use, and FIG. 5 is a state diagram of the combination between a safety cap for a disposable syringe according to an embodiment of the present invention and a syringe after use.

As illustrated in FIG. 4 and FIG. 5, after using the syringe 4, the safety cap 1 is pushed through the cutout insertion portion 2 from the lateral side of the upper portion of the syringe 4, so that the upper portion of the syringe 4 including the needle 4a is placed within the safety cap 1. In this case, when the fitting groove 3 of the safety cap 1 is press-fit into a protrusion 5 formed around the neck portion of the upper portion of the syringe 4, the safety cap 1 of almost the same size is forcibly coupled by the protrusion 5, and securely fit into the upper portion of the syringe 4.

When the safety cap 1 is forcibly coupled to the upper portion of the syringe by the fitting groove 3 as above, the safety cap 1 is hardly separated from the syringe 4, and thus the needle 4a can be safely stored even when it is arbitrarily separated from the syringe 4.

FIG. 6 is a perspective view of a safety cap for a disposable syringe before using the syringe according to another embodiment of the present invention.

As illustrated in FIG. 6, the safety cap for a disposable syringe according to another embodiment of the present invention is provided with a ball 16 at the inner upper end of the safety cap 10, and formed with a cutout insertion portion 14 in communication with the outside so that the needle is coupled to the upper end portion of the syringe 20 by inserting the needle 12 into the ball and rotating it.

The safety cap 10 is formed in the shape of a cone or a prismoid, and preferably the lower end portion is formed to be greater than the diameter of the upper end portion of the syringe 20. This is to reduce the pricking to a sharp tip of the needle 12 by allowing the fingers and hand to be positioned to deliberately make contact with the surface of the lower end portion of the safety cap 10 when preparing to mount the needle 12 in the safety cap 10. Meanwhile, in case the safety cap 10 is a polygonal prismoid, preferably, the prismoid is formed as a polygon of any one of a triangle, quadrangle, pentagon, hexagon and octagon.

Here, the cutout insertion portion 14 is formed at one side of the safety cap 10 as in the safety cap 1 of the one embodiment, and the width and length of the cutout insertion portion 14 are formed in a length corresponding to the diameter and length of the needle 12, so that the needle 12 can pass through easily. When the width and length of the cutout insertion portion 14 do not correspond to the diameter and length of the needle 12 and are larger, there is a higher possibility for the needle 12 to be exposed to dust, germs, etc., and thus it is not hygienic. Thus, preferably, it is formed in a size corresponding to the needle 12 as mentioned above.

Hereinafter, the state of use of a safety cap for a disposable syringe according to another embodiment of the present invention configured as above will be explained.

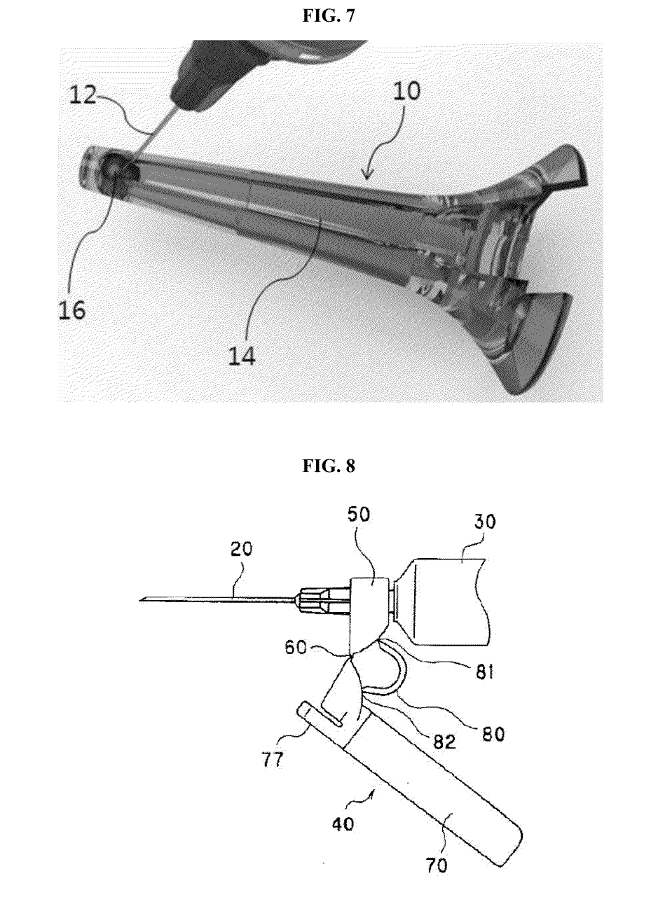

FIG. 6 is a front view of a safety cap for a disposable syringe before using the syringe according to another embodiment of the present invention, and FIG. 7 is a state diagram of the use of a safety cap for a disposable syringe according to another embodiment of the present invention.

FIG. 6 illustrates a disposable syringe before being used, where the needle 12 is not attached to the ball 16, and the safety cap 10 is coupled to be slightly apart from the syringe 20.

FIG. 7 illustrates the state where a needle is inserted into the ball after using the disposable syringe. The role of the ball 16 is to couple a needle to the safety cap by inserting the needle 12 into the ball 16 and rotating the ball 16 inserted with the needle 12 to the cutout insertion portion 14 side of the safety cap after using the disposable syringe 20. After using the syringe 20 as above, when the safety cap 10 is coupled to the needle 12, unlike FIG. 6, since the needle 12 is inserted into the ball 16, it can be found that the safety cap 10 gets close to the syringe 20.

Thus, it prevents a user, after using the syringe 20, from having a wound by being pierced by the needle 12 of the syringe 20 or from being infected by germs through the wound. Here, preferably, the ball 16 may be formed from a silicone or rubber material so that the needle 12 may be inserted easily.

Meanwhile, the inside of the ball 16 is formed to be empty. This causes part of the ball 16 to be inserted into the needle 12 when the needle 12 is inserted into the ball 16, and part of the inserted ball 16 blocks the inside of the needle 12. This causes part of the inserted ball 16 not to absorb the syringe liquid, thereby preventing reuse of needles when absorbing syringe liquid using the syringe 20 for reuse.

Also, preferably, the color of the safety cap for a disposable syringe according to another embodiment of the present invention may be a greenish color, which is a complementary color of red, so as to give psychological stability and reduce anxiety or hatred by the color of blood when blood smeared on the needle 12 is smeared on the safety cap 10 after use. According to the use of the syringe 20, the color of the safety cap 10 may vary to a color of any one from blue, yellow, grey and brown.

The safety cap for the disposable syringe according to the present invention was explained with reference to the exemplified drawings, but the present invention is not limited to the embodiments and drawings disclosed in the present specification, and various modifications may be made by those having ordinary skill in the art within the scope of technical idea of the present invention.

Since the needle of the syringe is covered after use by inserting the needle from the lateral side through the cutout insertion portion formed in the safety cap, the present invention can be useful as a safety cap for a syringe where there is hardly any danger of accidents having fingers pricked by the needle of the syringe, and the syringe can be safely disposed after use.

Also, since the safety cap is firmly fit in the syringe after use by the fitting groove formed in the safety cap, it can be useful by allowing the needle to be safely protected by the safety cap even when the needle is separated from the syringe.

Also, because safety caps have a simple structure considering the property of disposable syringes which have to be manufactured at low cost, it can be useful as a safety cap for a syringe where the manufacturing cost can be greatly reduced as compared to the conventional safety syringe.

Also, since a cutout insertion portion is formed at one side of the safety cap for the disposable syringe, and a ball is provided at the inner upper end of the cutout insertion portion so that a needle of the syringe is coupled to the safety cap by inserting the needle of the syringe into the ball and rotating it, it can be useful as a safety cap for a syringe preventing a user, after using the syringe, from having a wound by being pierced by the needle or from being infected by germs through the wound.

Also, since the inside of the ball is empty, when inserting the needle of the syringe into the ball, part of the ball is inserted into the needle of the syringe, and thus it can be useful as a safety cap for a syringe blocking the tip of the needle and preventing reuse of needles.

1. A safety cap for a disposable syringe coupled to an upper end portion of the main body of the syringe for covering a needle at the upper end portion, characterized in that the safety cap is formed with a cutout insertion portion cut out in the longitudinal direction so as to have an upper portion of the syringe including a needle inserted from the lateral side, and a fitting groove with a predetermined width in the longitudinal direction is formed by being partially cut out in the circumferential direction at a lower end portion formed with the cutout insertion portion.

2. The safety cap according to claim 1, characterized in that the safety cap is lifted up in the vertical direction and separated from the syringe when using the syringe, and after using the syringe, the safety cap covers the needle by having an upper portion of the syringe inserted from the lateral side through the cutout insertion portion and simultaneously a protrusion formed around the upper portion of the syringe is inserted and fit in a fitting groove formed at the lower end portion of the safety cap.

3. The safety cap according to claim 2, characterized in that the width in the longitudinal direction of the fitting groove of the safety cap is formed be slightly different from the thickness of the protrusion of the syringe, so that the protrusion is inserted in the fitting groove and the safety cap is forcibly coupled to the upper portion of the syringe.

4. The safety cap according to claim 1, characterized in that the safety cap is provided with a ball at the inner upper end portion, and a needle is coupled to the upper end portion of the main body of the syringe by inserting the needle into the ball through the cutout insertion portion at one side in communication with the outside and rotating the ball.

5. The safety cap according to claim 4, characterized in that the ball is made from a silicone or rubber material, empty in the inside.

6. The safety cap according to claim 4, characterized in that the safety cap is formed in the shape of a cone or a prismoid, and in the case of a polygonal prismoid, the prismoid is formed as a polygon of any one of a triangle, quadrangle, pentagon, hexagon and octagon, and the lower end portion is formed to be greater than the diameter of the upper end portion of the main body of the syringe.

7. The safety cap according to claim 6, characterized in that the safety cap is formed in a color of any one of green, blue, yellow, grey and brown, which gives psychological stability.

8. inner upper end portion, and a needle is coupled to the upper end portion of the main body of the syringe by inserting the needle into the ball through the cutout insertion portion at one side in communication with the outside and rotating the ball.

9. The safety cap according to claim 3, characterized in that the safety cap is provided with a ball at the inner upper end portion, and a needle is coupled to the upper end portion of the main body of the syringe by inserting the needle into the ball through the cutout insertion portion at one side in communication with the outside and rotating the ball.
